(19) United States
(12) Patent Application Publication
(10) Pub. No.: US 2009/0182292 A1
(43) Pub. Date: Jul. 16, 2009

(54) Medical instrument

(75) Inventors: Walter Egle, Koblach (AT); Mathias Wirbel, Braz (AT)

Correspondence Address: WENDEROTH, LIND & PONACK, L.L.P., 1030 15th Street, N.W.,, Suite 400 East, Washington, DC, 20005-1503, US

(73) Assignee: A.M.I. Agency of Medical Innovations

(21) Appl. No.: 12/318,848

(22) Filed: Jan. 9, 2009

(30) Foreign Application Priority Data
Jan. 10, 2008 (AT) A 34/2008

(51) Int. Cl. A61M 1/00; A61B 1/018

(52) U.S. Cl. 604/327; 600/106

(57) ABSTRACT

Medical instrument includes an endosurgical extraction bag for the extraction during a surgical procedure of material to be removed from a body on which surgery is to be performed, and a net disposed within the extraction bag. For the purpose of reducing the material to pieces, the net can be pulled through the material.

### BACKGROUND OF THE INVENTION

a) Field of the Invention

The invention relates to a medical instrument with an endosurgical extraction bag for the extraction during an endoscopic surgical procedure of material to be removed from a body on which surgery is to be performed.

b) Description of Related Prior Art

Medical instruments for the extraction of material, for example the gall bladder, to be removed during endoscopic surgery are known in various versions. In a known version a bag (=“endosurgical extraction bag”) is held by means of a hoop of a spring-elastic material at the distal end of a shaft of the instrument. In the state in which the instrument is delivered, the bag is rolled up and is located, together with the pressed-together hoop, in an enveloping tube encompassing the shaft. The shaft is introduced in this state through a trocar. When the enveloping tube on the shaft is withdrawn in the direction toward the proximal end of the shaft, the hoop and the bag appended thereon are released at the distal end of the enveloping tube, such that the bag can become unrolled and the hoop can unfold forming a fill opening of the bag. After the bag has been filled with the material to be extracted, it can be closed, for example by sliding the enveloping tube distally again on the shaft and the distal end of the enveloping tube pushes the bag together on the hoop, with the hoop being gradually pressed together again. A further feasible closure of the bag can be attained by means of a so-called drawstring closure. Herein a loop guided in a conduit of the bag, which encompasses its fill opening in this region, is drawn together whereby the bag is also torn off the spring-elastic hoop.

One disadvantage of these known extraction instruments comprises that, in general, the material to be extracted is of too great a size for it to be pulled out through the trocar through which the instrument has been inserted. Pulling the instrument together with the trocar through the opening in the body wall through which the trocar has been introduced is frequently also only possible by enlarging the opening in the body wall. Another feasibility consists in partially opening the bag again and inserting a morcellator (=size reducer) to reduce the material located in the bag to pieces and subsequently to extract the reduced material by means of an aspiration device. This procedure is highly complex and labor intensive.

A surgical extraction instrument with an extraction bag appended on a shaft is disclosed, for example, in U.S. Pat. No. 6,409,733 B1. Further, U.S. Pat. No. 5,769,794 A describes a separate extraction bag which is not secured in position on a shaft. A further extraction bag secured on a shaft is disclosed in U.S. Pat. No. 5,853,374 A, wherein this extraction bag comprises an opening directed toward the distal end of the instrument, which opening is closable.

### SUMMARY OF THE INVENTION

The invention addresses the problem of providing a medical instrument through which the extraction is facilitated of material, in particular organic material, to be removed from a human or animal body during a surgical procedure, in particular an endoscopic surgical procedure. According to the invention, this is attained through a medical instrument comprising an endosurgical extraction bag for the extraction of material to be removed during a surgical procedure from a body on which surgery is to be performed, and a net disposed within the extraction bag. The net can be pulled through the material for the purpose of reducing the material to pieces.

In the case of the medical instrument of the invention a net is disposed in the extraction bag which serves for reducing the material to be extracted. For this purpose the net is pulled through the material, in the process of which the filaments of the net cut through the material and herein cut the material in accordance with the mesh size of the net. The net can thus also be referred to as a cutting net. The material thus reduced can subsequently be removed from the body, for example by means of an aspiration line integrated into the medical instrument.

The net is preferably formed in the shape of a bag or it forms a portion of a bag, i.e. at least a segment of the wall of the bag is formed by a net. The bag formed by the net or including the net is initially disposed in the extraction bag when the material to be extracted is placed into the extraction bag. The material to be extracted, consequently, is placed into the bag formed by the net or including the net. The material to be extracted is held in this bag formed by the net or including the net until the net has been pulled through the material whereby the material is reduced to pieces.

In an advantageous embodiment of the invention the extraction bag is held by a shaft of the medical instrument, preferably by means of a hoop comprised of a spring-elastic material, which, without the action of an external force, unfolds annularly (for example approximately in the shape of a circular ring or an oval) such that a fill opening of the extraction bag is opened. The extraction bag is herein held over a large portion of its circumference on the hoop in the proximity of its fill opening. The bag formed by the net or including the net is located within the extraction bag and has an opening directed into the same direction as the fill opening of the extraction bag. The bag formed by the net or including the net is preferably substantially in contact on the wall of the extraction bag, at least in the region of the fill opening, in order to form as large an overall fill opening as is possible.

To close the extraction bag, preferably at least one draw element engages on the extraction bag, which draw element can be pulled in the direction toward the proximal end of the medical instrument by means of an actuation part. The draw element can herein extend through a conduit of the extraction bag in the proximity of its fill opening. When the draw element is pulled in the direction of the proximal end of the medical instrument, the extraction bag is gradually torn off the hoop and progressively closed. It can herein advantageously be pulled over a distal end section of the shaft, wherein at this distal end section a channel opens out which forms an inner hollow volume of the shaft. In the closed state of the bag, consequently, the distal end section of the shaft projects into the extraction bag through a residual opening of the constricted fill opening of the extraction bag. By means of a further draw element the bag formed by the net or including the net can be pulled into the hollow volume preferably opening out at the end side at the distal end section of the shaft, wherein the net is pulled through material initially disposed in the net while reducing it to pieces.

In the present document the specifications “proximal” and “distal” refer to the position with respect to the user (=surgeon). Consequently, a distal end is remote and facing away from the user and a proximal end is facing toward the user.

### BRIEF DESCRIPTION OF THE DRAWING

Further advantages and details of the invention will be explained in the following in conjunction with the attached drawings.

In the drawings:

FIG. 1 is an oblique view of an embodiment of a medical instrument according to the invention, in the state in which it is delivered,

FIG. 2 is an oblique view of the instrument of FIG. 1 after the outer enveloping tube has been retracted (=in the state in which the medical instrument can be filled),

FIG. 3 is an enlarged oblique view of a portion of the instrument in the state of FIG. 2, the extraction bag and the net sectioned centrally, a material to be extracted drawn schematically,

FIG. 4 is a distal section of the instrument from a viewing angle changed relative to FIG. 3, the extraction bag and the net cut open over an angular range of 90□,

FIG. 5 is an oblique view corresponding to FIG. 4, however without the extraction bag and the net,

FIGS. 6 and 7 are depictions corresponding to FIGS. 2 and 3, shortly after the start of the closing of the extraction bag,

FIGS. 8 and 9 are depictions corresponding to FIGS. 6 and 7, however with the extraction bag completely closed,

FIGS. 10 and 11 are depictions corresponding to FIGS. 8 and 9, however with the net partially retracted into the channel of the shaft,

FIGS. 12 and 13 are depictions corresponding to FIGS. 10 and 11, however with the net completely retracted into the channel of the shaft,

FIGS. 14 and 15 are depictions corresponding to FIGS. 12 and 13, however with an aspiration line inserted into the extraction bag.

The Figures are drawn to different scales.

### DETAILED DESCRIPTION OF THE PREFERRED EMBODIMENTS

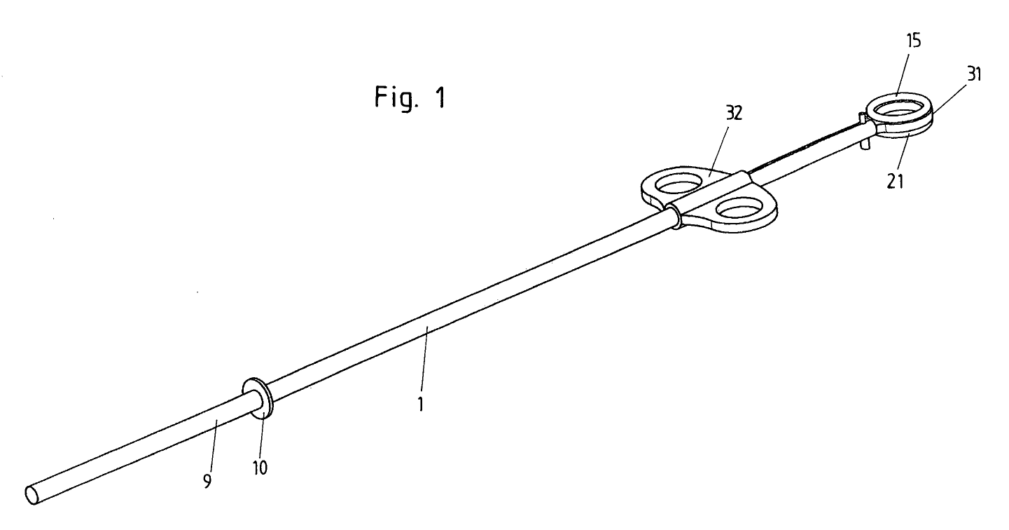

An embodiment of a medical instrument according to the invention is depicted in FIGS. 1 to 15. The instrument comprises a shaft 1, in the region of the distal end of which an endosurgical extraction bag 2 is held. The extraction bag 2 has a closed end 3 and a fill opening 4. The extraction bag 2 is fastened on the shaft 1 by means of a hoop 5 of a spring-elastic material. For this purpose the two ends of the hoop 5 are fastened on the shaft and the hoop 5 extends through a conduit 6 in the extraction bag 2, which conduit encircles the fill opening 4 in its proximity. Without an external force acting on it, the hoop 5 extends annularly, wherein it opens the fill opening 4 of the extraction bag 2.

A grip 32 can be disposed proximally on shaft 1.

In the extraction bag 2 is disposed a net 7 which has the form of a bag (=it forms a bag). Thus, the bag formed by the net 7 is located within the extraction bag 2, the bag formed by the net 7 having a closed end 8, which is located on the same side as the closed end 3 of the extraction bag 2, and an open end, which is located on the same side as the fill opening 4 of the extraction bag 2. When the medical instrument is in the state in which it can be filled (cf. FIG. 2 to 4), the bag formed by net 7 is in contact with the inner wall of the extraction bag 2 at least in the region of the fill opening of the extraction bag 2. Thereby as large an overall fill opening as possible is formed and the material is prevented from falling into the region between the net 7 and the extraction bag 2. The bag formed by net 7 is preferably substantially completely or at least largely in contact with the inner wall of the extraction bag 2 in order to form as large an overall filling volume as possible.

In the delivery state of the medical instrument (cf. FIG. 1) the extraction bag 2, and thus also the net disposed within the extraction bag 2, is located within the interior of an enveloping tube 9. The enveloping tube 9 is disposed such that it is displaceable on the shaft 1. The hoop 5 is pressed together and the extraction bag 2 with the net 7 disposed within it is rolled up.

When the enveloping tube 9 is slid on shaft 1 in the direction toward the proximal end 31 of the instrument, it gradually releases the hoop 5 with the extraction bag 2 held by it. After the enveloping tube 9 has been completely slid off the hoop 5 (cf. FIG. 2), the hoop 5 has spread open and the extraction bag 2, together with the bag formed by net 7 disposed within the extraction bag 2, can become unrolled (FIG. 2).

For the displacement of the enveloping tube 9, an outwardly projecting projection 10 can be disposed thereon, which, when the instrument is inserted into the trocar, comes to rest on the edge of the trocar whereby the enveloping tube 9 is displaced proximally during the continuing displacement.

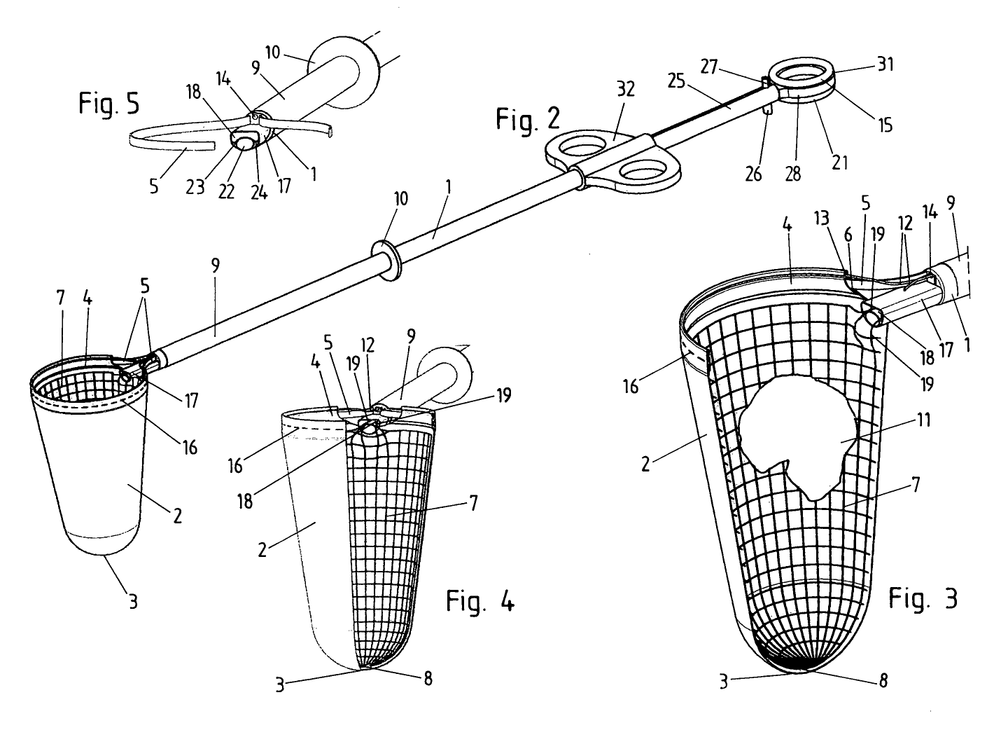

In the filling state depicted in FIGS. 2 to 4, in which the fill opening of the extraction bag 2 is opened and, with it, the bag formed by net 7 is opened and forms a common overall fill opening, during an endoscopic surgical procedure material 11 to be removed from the body can be placed into the extraction bag 2 and into the bag formed in it by net 7. Such material 11 is schematically drawn in FIG. 3. The material 11 is herein not depicted at the end of the extraction bag 2 located in FIG. 3 at the bottom, since the extraction bag can, for example, also be disposed horizontally in the body, i.e. the fill opening 4 does not face upwardly but laterally.

The material 11 can in particular be organic material. Such material to be removed from the body within the scope of endoscopic surgery is also referred to as resected tissue or specimen.

The fill opening 4 of extraction bag 2 can be drawn together. For this purpose a longitudinally extended draw element 12, for example a string or a cord or a band, is guided through a conduit 13 encircling the extraction bag 2 in the proximity of its fill opening 4. After their passage through the conduit 13, the two ends of the draw element 12 are inserted into a channel 14 in shaft 1 and extend through it and, by means of an actuation part 15, can be displaced with respect to shaft 1. Hereby the loop formed by the draw element 12 and extending through the conduit 13 is tightened whereby the extraction bag 2, starting from a distal section of hoop 5 opposite the shaft 1, is progressively torn off the hoop 5. During this tearing-off conduit 6 is torn open. To make possible or to facilitate this tearing-open, a weakened line-shaped zone or tear seam 16 is preferably formed in the material of the extraction bag 2 which delimits the conduit 6. This tear seam 16 extends preferably in the region of the outside of hoop 5.

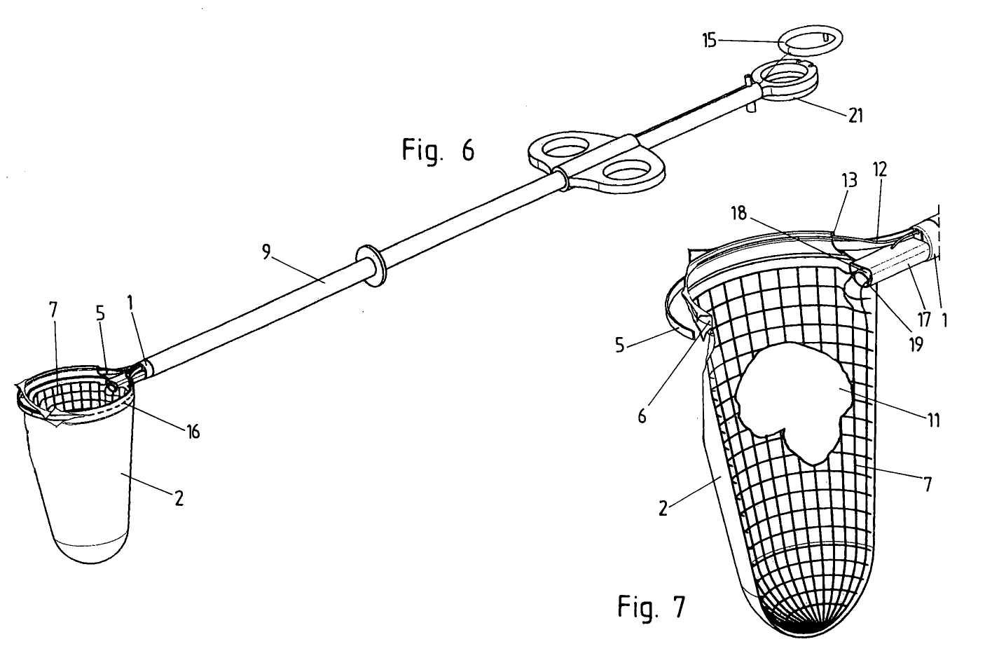

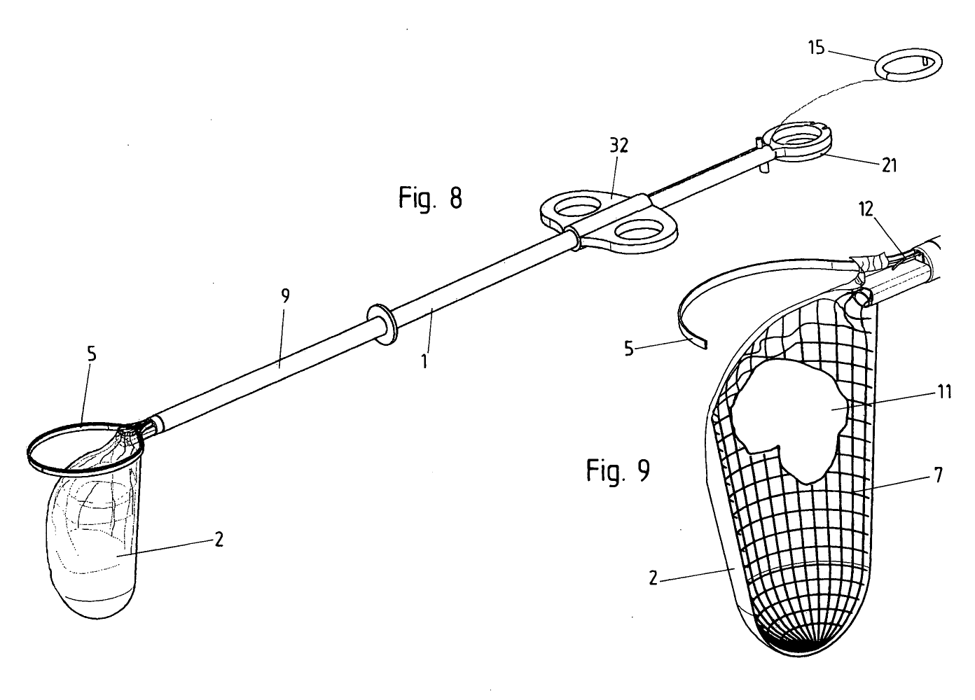

FIGS. 6 and 7 depict a state at the beginning of the tearing-off of the extraction bag 2 from hoop 5. In FIGS. 8 and 9 the final state of the closing of the extraction bag 2 is depicted. The loop formed by draw element 12 is herein largely tightened (its diameter measured in the direction of the longitudinal extent of shaft 1 has decreased to less than one third, preferably less than one fifth of the original value) and the extraction bag 2 has largely been torn from hoop 5 (over more than 80% of the original length of conduit 6). To prevent the extraction bag 2 from being completely torn off the hoop 5, the tear seam 16 can, for example, end at the desired final tearing site.

In the present state of the extraction bag 2 in which it is closed, the fill opening 4 of the extraction bag 2 is constricted and pulled over an end section 17 of shaft 1. The end section 17 of shaft 1 consequently projects into the extraction bag 2 through its constricted fill opening and the remaining fill opening 4 (residual opening) is filled out by the end section 17 of shaft 1 projecting into it and is thus closed.

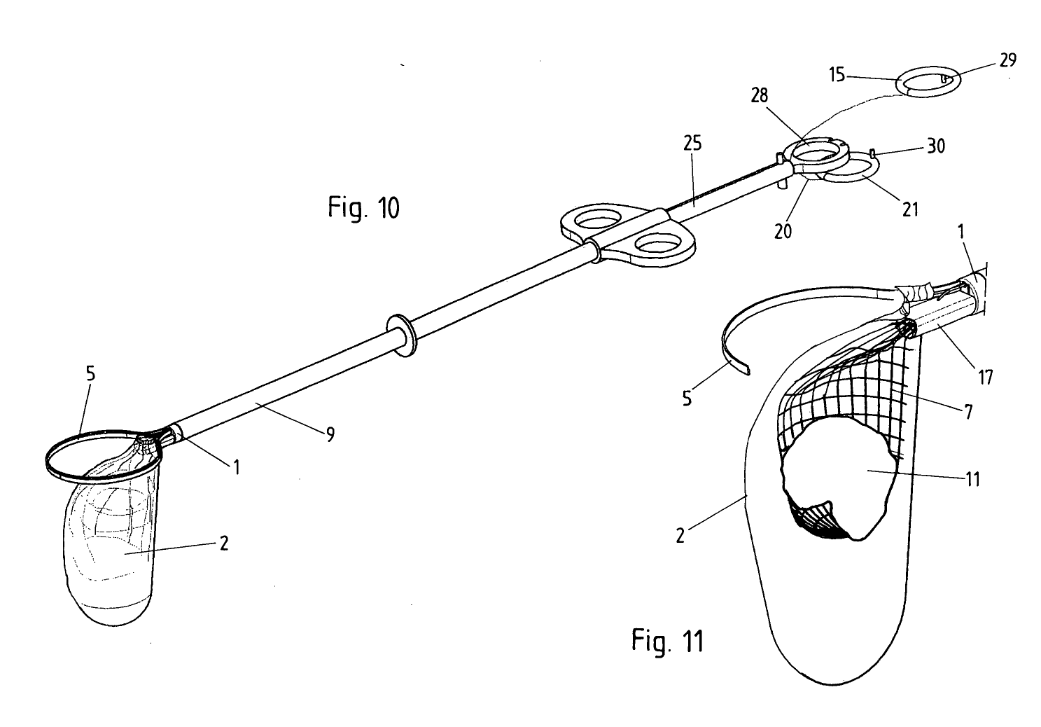

A channel opens out at the front end of the distal end side of end section 17 of shaft 1, which channel forms an inner hollow volume 18 of shaft 1 as is most clearly evident in FIG. 5. In the region in which it opens out the hollow volume 18 has preferably a diameter or an inner width of less than 0.5 cm. In this hollow volume 18 extends at least one longitudinally extended draw element 19 engaging on net 7. In the depicted embodiment several such draw elements 19 are provided (cf. in particular FIG. 4), which are formed by lengthened filaments of net 7. The draw elements 19 are attached within the hollow volume 18 on a common draw element 20, which can be displaced relative to the shaft 1 by means of an actuation part 21. The draw elements 19 could also extend up to the actuation part 21. Thereby the net 7 can be gradually drawn into the hollow volume 18 of shaft 1 by means of the actuation part 21. First, the bag formed by the net is herein closed so that the portion of this bag having the opening is drawn into the hollow volume 18. The portion of the bag formed by net 7 projecting from the hollow volume 18 is thus progressively decreased. In FIGS. 10 and 11 an intermediate position is depicted in which the net is partially drawn into the hollow volume 18.

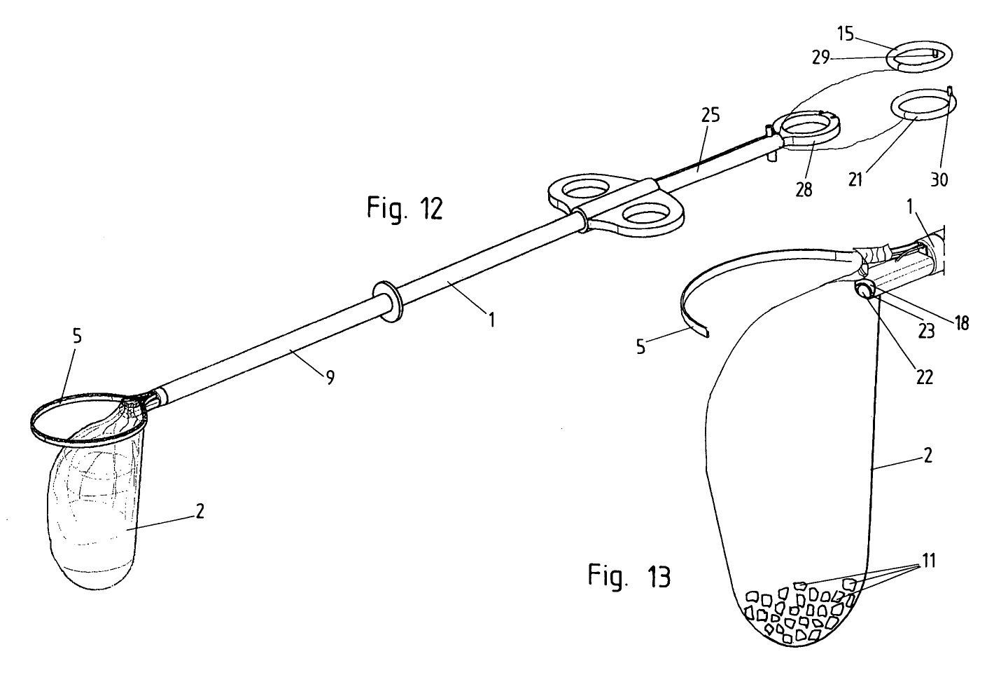

After the net 7 has been drawn so far into the hollow volume 18 that the material 11 in the bag formed by net 7 cannot be drawn further in the direction toward the distal end of the end section 17 and, due to its dimensions, can also not be drawn into the hollow volume 18, the filaments of net 7, when the net 7 is further drawn into the hollow volume 18, start cutting through the material 11 whereby the cut up parts of material 11 are pressed through the mesh of the net. After the net 7 has been completely drawn into the hollow volume 18, cf. FIGS. 12 and 13, the net has been pulled through the material 11 and the material 11 is correspondingly reduced to pieces.

In its final position the net 7, which in its initial position was located in the extraction bag 2, is completely pulled out of the extraction bag 2. Instead of drawing the net into a hollow volume 18 in shaft 1 or into a part connected with it, it would also be conceivable and feasible, for example, to pull the net through the opening of a ring connected with the shaft 1, wherein it is pulled out of the extraction bag 2.

In the depicted embodiment the end section 17 adjoins via a step the portion of the shaft located further proximally, which portion has a greater diameter compared to the end section 17. Into the step opens out the channel 14 for the draw element 12. Other formations are also conceivable and feasible.

The reduced parts of the material 11 can subsequently be aspirated by means of an aspiration line 22. The aspiration line 22 is initially located in a further channel 23 in shaft 1, which channel opens out at the distal end side of the distal end section 17 of shaft 1. The channel 23 and the channel forming the hollow volume 18 can be delimited against one another by a wall 24 (cf. FIG. 5).

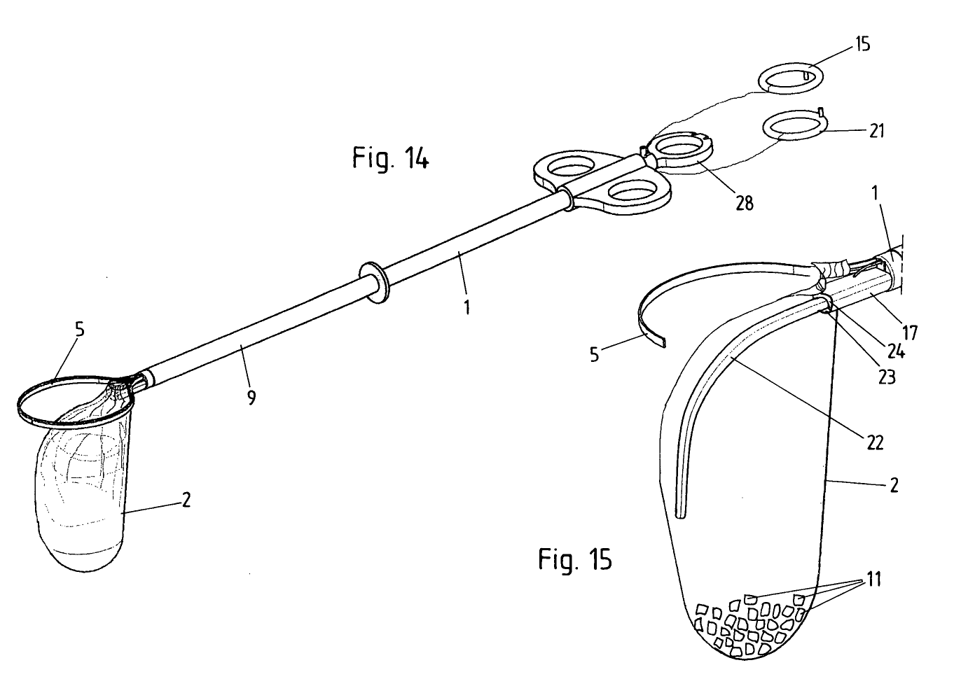

The aspiration line 22 can be slid out of the channel 23 as is depicted in FIGS. 14 and 15. For example, for this purpose a tube section 25, to which the aspiration line 22 is connected or through which the aspiration line 22 is guided, can be slid into a proximal end of shaft 1. At least the section of aspiration line 22, which can be slid out of the distal end of shaft 1, is comprised of a flexible material.

To make possible the irrigation of the closed extraction bag 2 with liquid or also gas, at least one irrigation channel, in addition to the at least one aspiration channel, can preferably be integrated into the aspiration line 22. Connection fittings 26, 27 for the at least one aspiration channel and the at least one irrigation channel are shown schematically.

The extraction bag 2 can thus be completely or at least partially emptied by alternating aspiration and irrigation. The extraction bag 2, completely or at least partially emptied in such a manner, can be pulled through the trocar without any problem or be removed from the body together with the trocar.

The filaments of net 7 are formed of a thin (such that it is possible to cut easily through the material 11) and adequately tear-resistant flexible material. The net is, for example, comprised of metal or nylon filaments or of carbon fibers.

The mesh size of net 7 is preferably less than 1 cm.

The extraction bag 2 is comprised of a suitable flexible material, for example of polyurethane, such as is known of conventional extraction bags.

It would also be conceivable and feasible that the net 7 forms only a portion of the bag disposed within the extraction bag 2, for example only the bottom in the region of the closed end 8 of this bag.

Net 7 could also be drawn into several hollow volumes 18 in the shaft or into a part connected with it (while undoing the net) or through several rings connected with the shaft.

A separate device for closing the bag formed by net 7 or including this net could also be provided. For example, a draw element which extends through a conduit of this bag encompassing the bag in the proximity of its fill opening could be provided.

The aspiration line 22 (or aspiration-irrigation line 22) integrated into the medical instrument could also be omitted and the reduced material 11 could be aspirated from the extraction bag 2 by means of a separate aspiration instrument.

In a further feasible embodiment of the invention the extraction bag 2 could also be provided in the proximity of its closed end 3 with an extension having a decreased diameter. The material reduced by means of net 7 could fall into this “worm-like” extension. This worm-like extension could herein have so small a diameter that the medical instrument with the reduced material fallen into this extension can be drawn out through the trocar or be withdrawn from the body together with the trocar.

The actuation parts 15, 21 for the extraction bag 2 and the net 7 are shown in the depicted embodiment example in the form of rings. In their initial position they are held on a proximal ring 28 disposed on tube section 25, for example snapped into it by means of pins 29, 30. These actuation parts 15, 21 can also be formed differently in another manner, for example in the form of actuation levers or rotary grips. Formations different for the actuation part 15 and for the actuation part 21 are also feasible, for example in order to be able to exert a greater force for drawing back the net 7. The actuation parts 15, 21 can be disposed on shaft 1 or on a part rigidly connected with it or—as in the depicted embodiment example—displaceably connected with.

It would also be conceivable and feasible that the at least one draw element 19 for drawing the bag, formed by net 7 or including such, out of the extraction bag 2 does not extend through the fill opening 4 of the extraction bag 2. The extraction bag 2 could have a separate lateral opening through which this at least one draw element 19 extends and through which the bag formed by net 7 or including such is drawn out.

It is further conceivable and feasible not to close the extraction bag 2 using a draw element 12 but rather by sliding the enveloping tube 9 again in the distal direction, wherein the extraction bag 2 is pushed together on the hoop 5 and the fill opening 4 is closed, as is known in the case of extraction bags of prior art.

The medical instrument according to the invention can be utilized in different types of endoscopic surgical procedures for the extraction of material from the human or animal body (=removal of material from the body such that, if possible, it does not come into contact with other body parts). These types of endoscopic procedures include, for example, laparoscopic surgery or intraluminal endoscopic surgery, which are also known by the term N.O.T.E.S. When used in such flexible endoscopic operations, the shaft as well as the parts distally disposed thereon can be formed such that they are flexible. The net 7 is suitable in the various types of endoscopic procedures for reducing material, in particular body material, of adequate softness.

In an instrument according to the invention with a net 7 disposed within the extraction bag for dividing material 11 collected in the extraction bag 2, the extraction bag 2 could also be formed in a manner other than described in the depicted embodiment example. The extraction bag 2, as is known in prior art, could for example also have a closable fill opening directed into a distal direction. The bag formed by net 7 or including net 7 disposed within the extraction bag could in this case also have a closable opening directed distally and be drawable from its proximal end into a hollow volume of the shaft.

As is evident in the above description, the scope of the invention is not limited to the depicted embodiment example but rather should be determined with reference to the attached claims together with its full range of feasible equivalents. While the preceding description and the drawing represent the invention, it is obvious to a person of skill in the art that various modifications can be carried out therein without leaving the true spirit and scope of the invention. Furthermore, the entire content of priority Austrian application A34/2008 is incorporated herein by reference.

### LEGEND TO THE REFERENCE NUMBERS

| | |
|---|---|
| 1 | Shaft |
| 2 | Endosurgical extraction bag |
| 3 | Closed end |
| 4 | Fill opening |
| 5 | Hoop |
| 6 | Conduit |
| 7 | Net |
| 8 | Closed end |
| 9 | Enveloping tube |
| 10 | Projection |
| 11 | Material |
| 12 | Draw element |
| 13 | Conduit |
| 14 | Channel |
| 15 | Actuation part |
| 16 | Tear seam |
| 17 | End section |
| 18 | Hollow volume |
| 19 | Draw element |
| 20 | Draw element |
| 22 | Aspiration line |
| 23 | Channel |
| 24 | Wall |
| 25 | Tube section |
| 26 | Connection fitting |
| 27 | Connection fitting |
| 28 | Ring |
| 29 | Pin |
| 30 | Pin |
| 31 | Proximal end |
| 32 | Grip |

1. Medical instrument comprising
an endosurgical extraction bag for the extraction of material to be removed during a surgical procedure from a body on which surgery is to be performed, and
a net disposed within the extraction bag, which net can be pulled through the material for the purpose of reducing the material to pieces.

2. Medical instrument as claimed in claim 1, wherein the net forms a bag or a portion of a bag.

3. Medical instrument as claimed in claim 2, wherein the bag, formed by the net or including the net, for the purpose of pulling the net through the material to be reduced can be pulled by means of at least one draw element engaging on the bag and an actuation part cooperating with the at least one draw element.

4. Medical instrument as claimed in claim 2, wherein the bag formed by the net or including the net is in an initial position within the extraction bag and, after the net has been pulled through the material to be reduced, is at least partially pulled out of the extraction bag.

5. Medical instrument as claimed in claim 2, wherein the bag, formed by the net or including the net, for the reduction of the material initially located in this bag, can at least be drawn partially into at least one inner hollow volume of the medical instrument.

6. Medical instrument as claimed in claim 5, wherein the at least one inner hollow volume is located in a shaft of the medical instrument, through which shaft extends the at least one draw element.

7. Medical instrument as claimed in claim 6, wherein on the shaft an enveloping tube is disposed which is displaceable from an initial position, in which the extraction bag is located within the enveloping tube, in the direction toward the proximal end of the medical instrument into a final position in which the extraction bag is located outside of the enveloping tube.

8. Medical instrument as claimed in claim 2, wherein in a filling state of the extraction bag for filling in the material to be extracted, a fill opening of the extraction bag is opened and the bag formed by the net or including the net is opened toward the same side as the extraction bag, wherein the extraction bag is closable.

9. Medical instrument as claimed in claim 8, wherein for closing the extraction bag by constricting the fill opening of the extraction bag a draw element extending in a conduit about the extraction bag in the proximity of its fill opening can be pulled together by means of an actuation part.

10. Medical instrument as claimed in claim 9, wherein the extraction bag in its filling state is suspended on a hoop comprised of a spring-elastic material and the extraction bag for closing the extraction bag can be progressively torn off the hoop starting from a distal section of the hoop by means of the at least one draw element.

11. Medical instrument as claimed in claim 8, wherein in the closed state of the extraction bag into a residual opening of the constricted fill opening of the extraction bag projects a distal end section of the shaft, wherein at this distal end section of the shaft at least one hollow volume opens out for drawing in the net.

12. Medical instrument as claimed in claim 1, wherein the medical instrument includes an aspiration line which, for aspirating material reduced to pieces by means of the net, can be introduced into the extraction bag.

13. Medical instrument as claimed in claim 12, wherein the aspiration line extends through a channel in the shaft and is displaceable with respect to the shaft.

14. Medical instrument as claimed in claim 13, wherein the channel receiving the aspiration line opens out at the end side of the distal end section of the shaft.

15. Medical instrument as claimed in claim 1, wherein the medical instrument is adapted for use in an endoscopic surgical procedure.
